(19) United States
(12) Patent Application Publication
(10) Pub. No.: US 2020/0187901 A1
(43) Pub. Date: Jun. 18, 2020

(54) ENHANCED ULTRASOUND SYSTEMS AND METHODS

(71) Applicant: The Regents of the University of California, Oakland, CA (US)

(72) Inventors: Preetham Suresh, La Jolla, CA (US); Danilo Gasques Rodrigues, La Jolla, CA (US); Nadir Weibel, La Jolla, CA (US); Elizabeth A. Anderson, La Jolla, CA (US)

(21) Appl. No.: 16/643,505

(22) PCT Filed: Aug. 31, 2018
(86) PCT No.: PCT/US2018/049273

§ 371 Date: Feb. 28, 2020

Related U.S. Application Data
(60) Provisional application No. 62/554,505, filed on Sep. 5, 2017.
(60) Provisional application No. 62/553,103, filed on Aug. 31, 2017.

(51) Int. Cl. A61B 8/00; A61B 8/08; G06T 7/00

(52) U.S. Cl. CPC A61B 8/4245; A61B 8/462; G06T 2207/10132; A61B 8/5207; G06T 7/0012; A61B 8/5215

(57) ABSTRACT

Systems, devices, and methods are disclosed for an enhanced ultrasound system. A system may include an ultrasound probe. The system may include processing circuitry communicatively coupled to the ultrasound probe. The system may also include an AR device receiving image information from the processing circuitry and displaying one or more ultrasound images from the ultrasound probe in a field of view of an operator.

### CROSS-REFERENCE TO RELATED APPLICATIONS

The present application is a U.S. National Phase under 35 U.S.C § 371 of International Application No. PCT/US18/49273, filed Aug. 31, 2018, which claims priority to U.S. Provisional Patent Application No. 62/554,505, filed Sep. 5, 2017, and U.S. Provisional Patent Application No. 62/553,103, filed Aug. 31, 2017, the contents of which are incorporated herein by reference in their entirety

### TECHNICAL FIELD

The present disclosure is generally related to ultrasound imaging. More particularly, some embodiments of the present disclosure are related to enhanced ultrasound imaging using augmented reality.

### Brief Description of the Embodiments

In some embodiments, an ultrasound imaging system includes an ultrasound probe; processing circuitry communicatively coupled to the ultrasound probe; and an AR (augmented reality) device receiving image information from the processing circuitry and displaying one or more ultrasound images from the ultrasound probe in the field of view of an operator.

In embodiments, the ultrasound probe includes a tracking mechanism to generate and send position information and orientation information of the ultrasound probe to the AR device, such that the one or more ultrasound images dynamically move as a position of the ultrasound probe changes.

In embodiments, the AR device further receives depth information corresponding to the one or more ultrasound image.

In embodiments, the ultrasound probe includes a first input, such that interacting with the first input changes a depth of the one or more ultrasound images displayed in the field of view of the operator.

In embodiments, the ultrasound probe includes a second input, such that interacting with the second input freezes one of the one or more ultrasound images.

In embodiments, the ultrasound probe further receives supplemental information and displays the supplemental information in the field of view of the operator.

In embodiments, the supplemental information includes one or more of patient information, treatment information, medication information, and a reference ultrasound image.

In embodiments, a given ultrasound image includes depth information for the given ultrasound image. The ultrasound image may also include position information for the given ultrasound image, such that the one or more ultrasound images and corresponding depth and position information are used to generate a 3D image of a region captured by the ultrasound probe.

In further embodiments, a method for ultrasound imaging includes receiving position and orientation information from an ultrasound probe; receiving image information on a current image captured by the ultrasound probe; identifying an imaging plane for the current image captured by an ultrasound probe based on the position and orientation information; retrieving a reference image from a database, the reference image corresponding to the imaging plane of the current image captured by the ultrasound probe; and sending the current image and the reference image to an AR device to display the current image and the reference image in an operator's field of view.

In embodiments, the current image is displayed in the operator's field of view such that the current image appears to project from a distal end of the ultrasound probe.

In embodiments, the current image is displayed adjacent to the reference image.

In embodiments, the ultrasound probe further receives supplemental information and displays the supplemental information in the operator's field of view.

In embodiments, the supplemental information includes one or more of patient information, treatment information, medication information, and a reference ultrasound image.

In embodiments, the supplemental information is overlaid onto the current image.

In still further embodiments, a method for ultrasound imaging includes receiving position and orientation information from an ultrasound probe; receiving image information on a current image captured by the ultrasound probe; identifying an imaging plane for a current image captured by the ultrasound probe based on the position and orientation information; sending the current image to an AR device to display the current image in a field of view of an operator; and determining a position of a target under a portion of skin of a subject.

In embodiments, the method further includes identifying an entry point and a corresponding trajectory from the entry point for a tool to reach the target. The method may include sending the entry point and the corresponding trajectory to the AR device to overlay an image of the entry point and the corresponding trajectory onto the current image in the field of view of the operator.

In embodiments, the method may further include receiving position and orientation information from the tool. The method may include sending the position and orientation information corresponding to the tool to the AR device to overlay an image of the tool onto the entry point and the corresponding trajectory overlaid onto the current image in the field of view of the operator.

In embodiments, the method may further include sending a position of the target to the AR device to display the position in the field of view of the operator.

In embodiments, the method may further include extracting depth information from the current image captured by the ultrasound probe.

In embodiments, the target is tissue.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

Various embodiments are disclosed herein and described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a diagram illustrating an example of an enhanced ultrasound imaging system in accordance with one embodiment of the technology described herein.

FIG. 2 is a diagram illustrating an example of an image displayed adjacent an ultrasound probe in accordance with one embodiment of the technology described herein.

FIG. 3 is a diagram illustrating another example of an image displayed adjacent an ultrasound probe in accordance with one embodiment of the technology described herein.

FIG. 4 is a diagram illustrating a process for displaying a reference image adjacent a captured image in accordance with one embodiment of the technology described herein.

FIG. 5 is a diagram illustrating a process for illustrating entry points and trajectories for a tool in accordance with one embodiment of the technology described herein.

FIG. 6 is a diagram illustrating another example of an image displayed adjacent a probe, along with an example of an AR device (AR Glasses) and a workstation.

FIG. 7 is a diagram depicting an example computing component used to implement features according to certain embodiments of the provided disclosure.

FIG. 8 is a diagram illustrating another example of an image displayed adjacent an ultrasound probe in accordance with one embodiment of the technology described herein.

FIG. 9 is a diagram illustrating another example of an image displayed adjacent an ultrasound probe in accordance with one embodiment of the technology described herein.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION OF THE EMBODIMENTS

Ultrasound imaging uses high-frequency sound waves to view the inside anatomy of a human body in real time. The ultrasound images can be produced by sending pulses of ultrasound waves into the tissue using a probe. When the ultrasound waves echo off the tissue, different tissues reflect varying degrees of sound, and the echoes can be recorded and displayed as an image. Thus, ultrasound imaging is a powerful medical tool that can help a physician or medical professional evaluate, diagnose, and treat medical conditions.

Currently, ultrasound imaging is performed to be displayed in 2D on a screen or monitor. As a result, the ultrasound imaging is limited to the constraints of the screen or monitor. Additionally, the operator is frequently required to remove his or her eyes from the subject to view the images displayed on monitor.

Embodiments of the systems and methods disclosed herein relate to enhanced ultrasound imaging systems and methods that can be used in a variety of applications including, for example, medical applications. In some embodiments, image information obtained using an analysis tool such as, for example, an ultrasound probe, can be provided to an AR device worn by the health-care practitioner operating the analysis tool. The AR device causes the captured image to be presented to the practitioner such that the image appears within the field of view of the practitioner. For example, in some embodiments, the ultrasound image can be caused to appear at the location of the analysis tool. For example, in the case of ultrasound imaging, the image can be generated using the AR device such that the image appears at, or near, the location of the ultrasound probe in real-time or near-real-time. Further embodiments can include a tracking system to determine and identify the position of the analysis tool (e.g., handheld ultrasound probe) in 3D space and the position information used to determine placement of the image such that it is in the proximity of the analysis tool. For example, in some embodiments, the image can be made to appear as if it is being projected by the analysis tool on to the body of the subject being analyzed.

Further embodiments can use enhanced ultrasound imaging technology to guide a health care practitioner in the performance of a medical procedure. For example, some implementations can be used to show the path of a surgical tool or other like implement that may be used subcutaneously. For example, embodiments can include position tracking to detect the positioning of the surgical implement. This position information can be provided to a processing system that can effectively overlay an image of the tool on the image presented by the AR device such that the system shows the path of the surgical tool as it is inserted into and manipulated within the subject. As another example, embodiments can be used to identify entry points and trajectories for a surgical tool and to display those to the healthcare practitioner during the performance of a procedure.

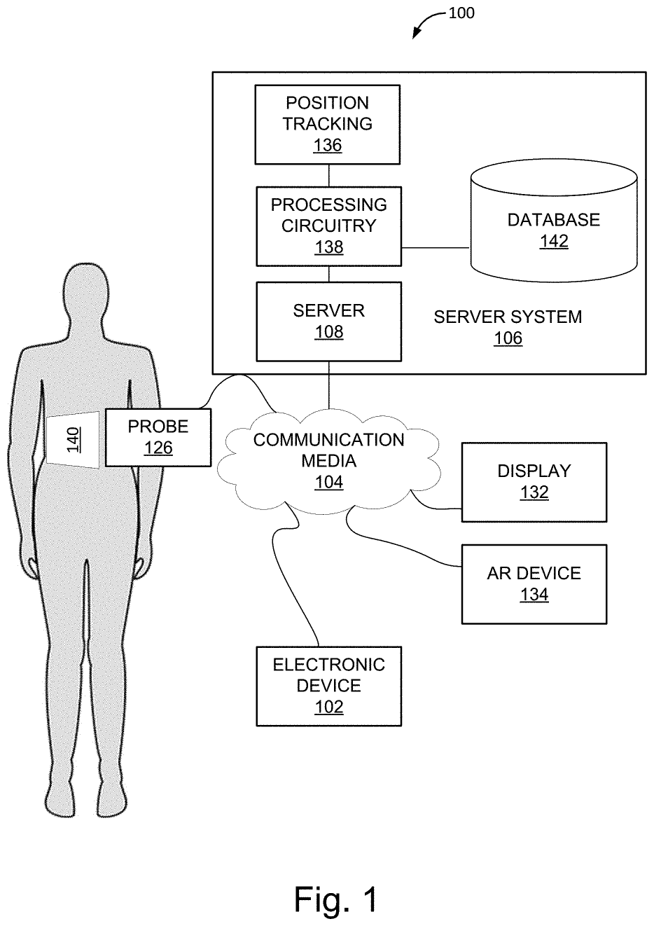

FIG. 1 is a diagram illustrating an example system for enhanced imaging in accordance with one embodiment of the technology described herein. Although this example is described in terms of an ultrasound imaging system, it should be appreciated how the systems and methods disclosed herein can be used with other imaging technologies beyond ultrasound imaging. In the illustrated example, an ultrasound probe 126 is used by an operator (e.g., a healthcare practitioner) to capture images of the subject. Although the subject is a human subject in the example illustrated in FIG. 1, other subjects can be used. The images captured by the ultrasound probe can be sent to a display 132. For example, display 132 can be a conventional display such as a stereoscopic display that is used for ultrasound imaging procedures.

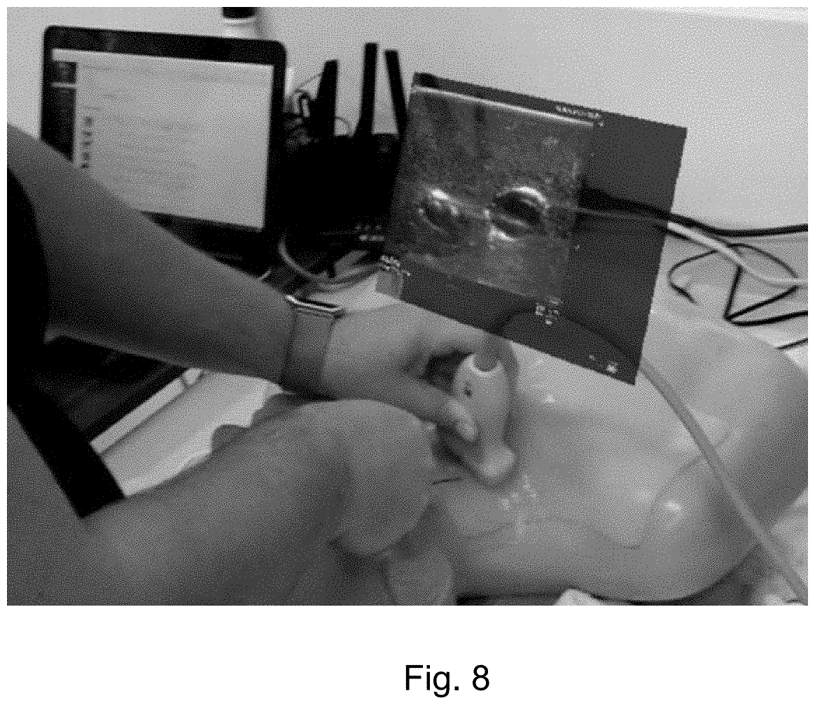

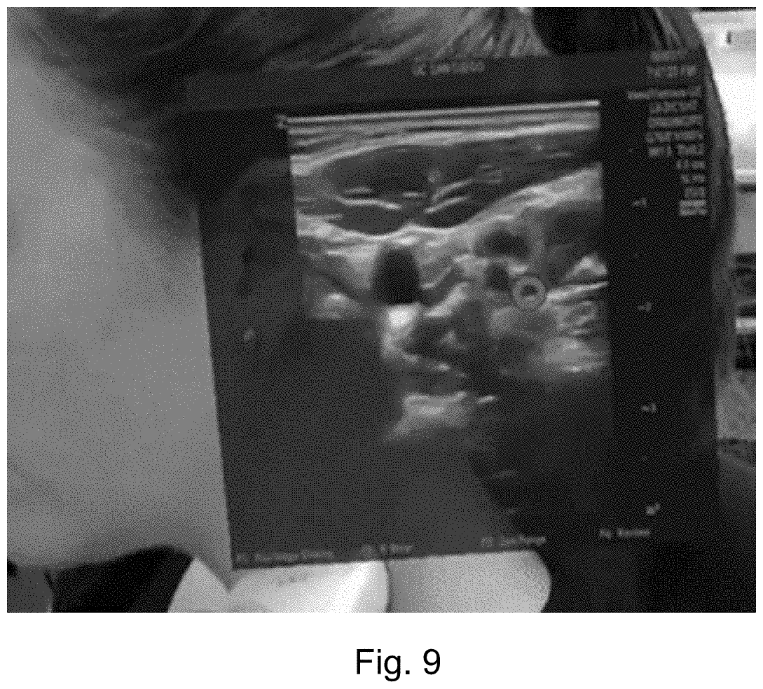

The images captured by ultrasound probe 126 can also be provided to an AR device 134 such that the images can be displayed in the field of view of the operator. For example, an AR device (e.g., AR glasses) can be provided to, and worn by, the healthcare practitioner. The captured images can be displayed on AR device 134 such that they appear in the field of view of the operator even if the operator is not looking in the direction of the physical display. For example, referring to FIGS. 8 and 9, the image may be in the operator's field of view. In FIG. 8, the plane of the image may appear to be parallel with the direction of the ultrasound waves coming from ultrasound probe 126. The image may appear above ultrasound probe 126, such that an operator can view the subject, ultrasound probe 126, and the image at the same time. In FIG. 9, the plane of the image may appear to be perpendicular to the direction of the ultrasound waves coming from ultrasound probe 126. It should be appreciated that the plane of the image may be adjusted by the operator using one of the one or more inputs, as described herein. The image may appear above ultrasound probe 126, as described in FIG. 8. Examples of an AR device that can be used to provide this display can include, for example, the Microsoft HoloLens, the Solos, the Merge Holo Cube, the Meta 2, the Epson Moverio, the Sony SmartEyeglasses, the HTC Vive®, the Zeiss Smart Glass, the Vuzix Smart Glasses, and other like AR devices.

Accordingly, in such embodiments, the operator does not need to look away from ultrasound probe 126 during a procedure to view a fixed physical display, such as display 132. Instead, the captured images can be displayed in the field of view of the operator while he or she is manipulating, and looking at, ultrasound probe 126. In embodiments, ultrasound probe 126 may have one or more inputs (not shown) on the probe itself. For example, a button, scroll wheel, touch pad, microphone, camera, or other feature on ultrasound probe 126 may be interacted with to affect the display on a device (e.g., electronic device 102, display 132, or AR device 134). For example, clicking a button may pause or freeze the display on a current image. Clicking the button again may unfreeze the display so that the images are presented dynamically as ultrasound probe 126 moves around.

The enhanced ultrasound imaging system can be configured to include position tracking circuitry 136 such that the position of the analysis tool can be determined in 3D space. In embodiments, position tracking circuitry 136 may also be able to determine an orientation of the analysis tool in 3D space. In some embodiments, embedded markers including transceivers to send and receive signals and DOF sensors using gyroscopes, accelerometers, or other sensors may be used to track a position and orientation of ultrasound probe 126.

The position and orientation may also be tracked through electromagnetic tracking systems, acoustic tracking systems, optical tracking systems, or mechanical tracking systems. Electromagnetic tracking may measure magnetic fields generated by running electric current through sets of wires, each arranged perpendicular to the other sets of wires. Acoustic tracking systems may emit and sense sound waves and determine a location based on a time taken to reach a sensor of the acoustic tracking system. Optical tracking systems may use light sources and cameras to determine a position and orientation. Mechanical tracking systems may require a physical connection between a fixed reference point and the moving object.

Any of a number of available position detection systems can be used to allow the enhanced imaging system to determine the position (and in some instances orientation) of ultrasound probe 126, as described herein. The position tracking circuitry 136 may receive or capture one or more signals from probe 126. For example, in some embodiments, VR tracking technologies can be used. As a further example, in one embodiment the HTC Vive® system can be used to determine the position of the analysis tool. In another example, a driveBay and trakSTAR unit may use electromagnetic tracking to determine a position and orientation of the analysis tool.

Processing circuitry 138 can be included to adjust the positioning of the ultrasound image as displayed by AR device 134 relative to ultrasound probe 126. For example, in some embodiments, processing circuitry 138 can use the determined position of ultrasound probe 126 to cause the ultrasound image to be displayed at AR device 134 such that it appears adjacent to the actual location of ultrasound probe 126. In the example illustrated in FIG. 1, the ultrasound images are displayed by AR device 134 such that it appears to be positioned on the patient at, or proximal to, the location of ultrasound probe 126. This is illustrated by image 140 adjacent probe 126.

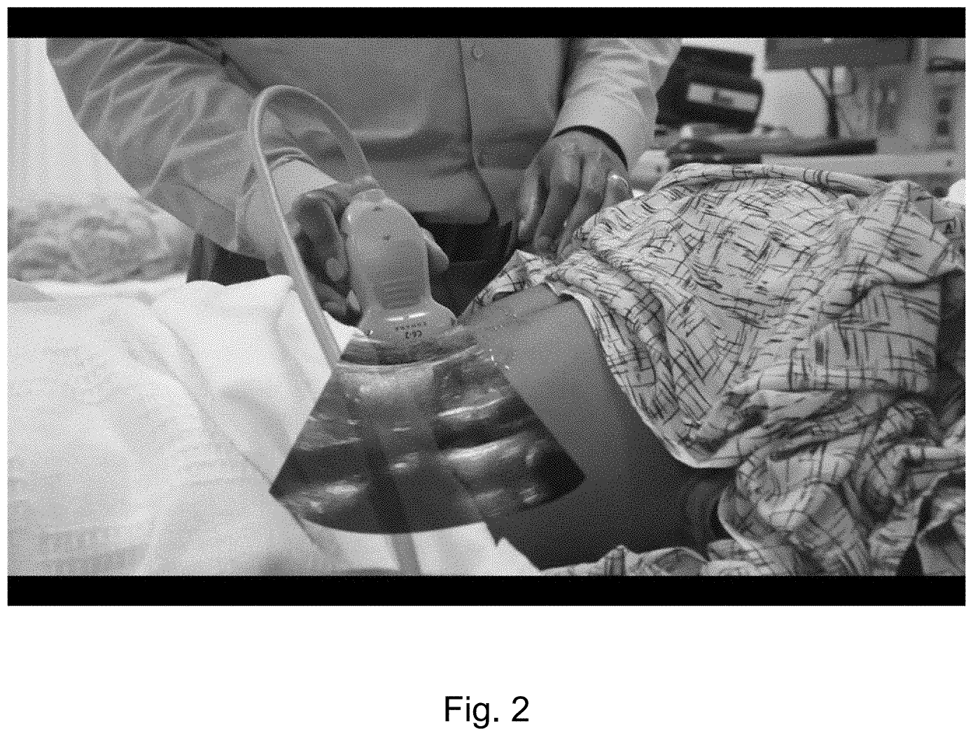

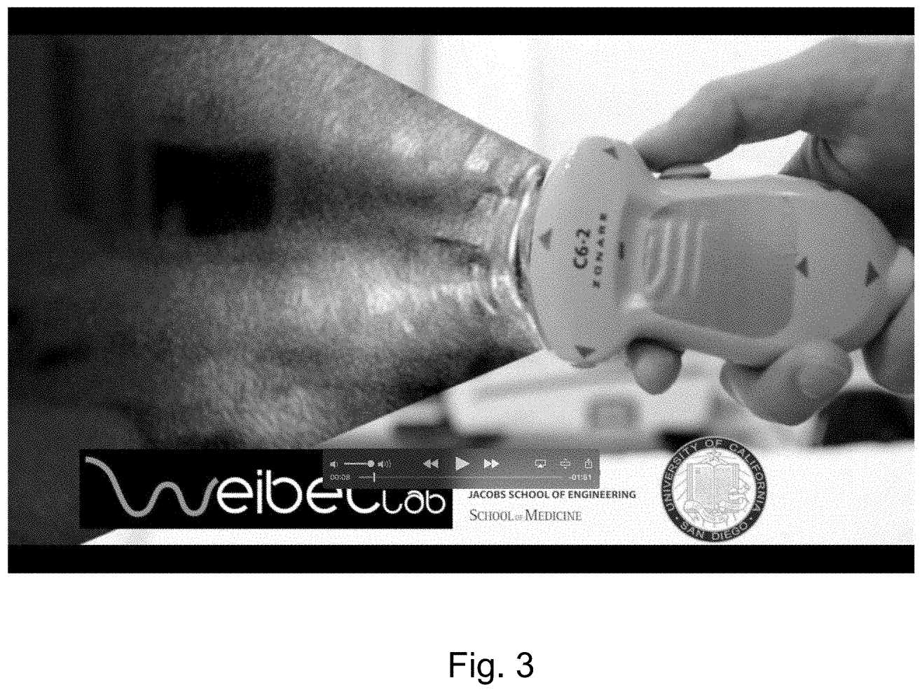

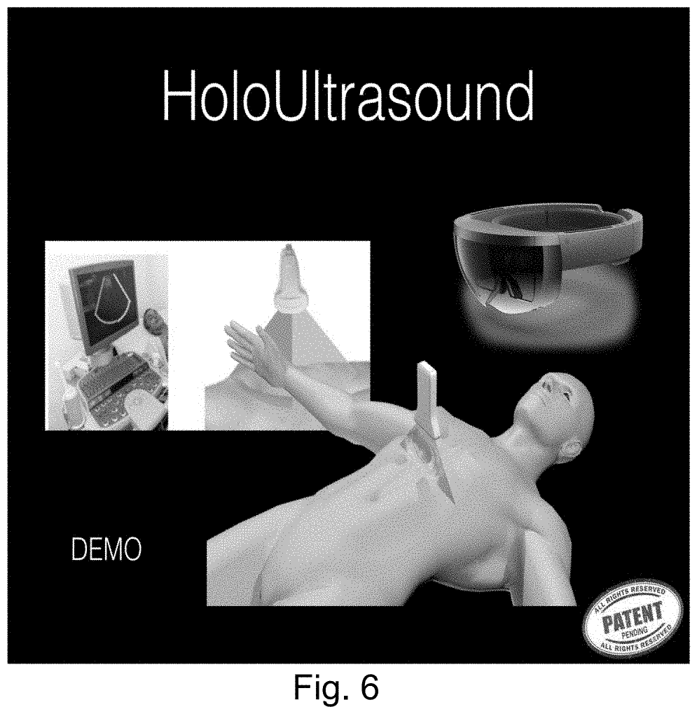

An example of this is illustrated in FIG. 2. In this example, the ultrasound image is made to appear in the field of view of the operator adjacent to the distal end of the ultrasound probe. Accordingly, as the operator is looking at and manipulating the ultrasound probe the operator can also see the ultrasound images being captured by the probe in real-time or near-real-time. FIG. 3 illustrates an example in which the ultrasound probe is held up and the image is repositioned at AR device 134 to appear as if it is being projected from the distal end of the probe. FIG. 6 illustrates another example of an image displayed adjacent a probe, along with an example of an AR device 134 (AR Glasses in this example) and a workstation.

In embodiments, depth information may be based on reflected waves of ultrasound probe 126. The reflected waves may be used to calculate a depth of a surface off of which the waves were reflected. For example, the time the reflected wave takes to return to ultrasound probe 126 may be used to determine a given depth of the surface. Because depth information can be included in ultrasound and other like imaging technologies, embodiments can be implemented such that the AR device 134 recreates the image to appear as a 3D image under the skin of the subject. Depth information determined for various features, or targets, within the image can be used to create the corresponding left eye and right eye stereo image in AR device 134 such that the user can perceive depth within the projected image and this image can be made to appear beneath the skin of the subject. This can provide a more realistic view to the operator as he or she is manipulating the ultrasound probe 126. In embodiments, an input on ultrasound probe 126 may be interacted with to adjust a depth of the image.

The example in FIG. 1 also includes a database 142, which can include one or more individual databases. In some embodiments, information from database 142 can be used in combination with AR device 134 to provide supplemental information with the images being displayed. For example, patient information, treatment information, medication information, and other information can be pulled from database 142 and displayed to the operator. In another embodiment, the database can include a plurality of reference scans that can be displayed to the operator along with the images captured by the ultrasound probe 146. In embodiments, an input on ultrasound probe 126 may be interacted with to display the reference scans or supplemental information adjacent to ultrasound probe 126. Interacting with the input may overlay the reference scans or supplemental information or switch the display from a current image to the reference scans or the supplemental information. As a further example of this, database 142 can include a plurality of images in the form of slices that are taken from reference CT scans. These individual slices can show, for example, scans taken from a “normal” subject, or they can be scans taken from a subject having one or more conditions (and hence, “abnormal” appearance of organs or other tissue). The position and orientation of ultrasound probe 126 can be determined such that the ultrasound system can determine the path of an image slice through the body. A corresponding slice from database 142 can be retrieved and shown adjacent to the image slice from the ultrasound system. The corresponding slice may be retrieved because of corresponding metadata indicating the slice is taken from around the same position and orientation ultrasound probe 126 is currently capturing. As the probe is moved, the new slice can be determined and the corresponding slice pulled from the database. In this way, the operator can be given the opportunity to compare visually, the real-time (or near-real-time) images from the subject with the corresponding reference images from the database. This can aid the operator in diagnosing various conditions or confirming the health of the subject.

As shown in FIG. 1A, environment 100 may include one or more of electronic device 102, display 132, AR device 134, and server system 106. Electronic device 102, display 132, and AR device 134 can be coupled to server system 106 via communication media 104. As will be described in detail herein, electronic device 102, display 132, AR device 134, and/or server system 106 may exchange communications signals, one or more images, user input, supplemental information, position information, orientation information depth information, metadata, and/or other information for electronic device 102, display 132, or AR device 134 via communication media 104. In some embodiments, server system 106 and electronic device 102 or AR device 134 may be packaged into a single component.

Electronic device 102 may include a variety of electronic computing devices, such as, for example, a smartphone, tablet, laptop, wearable device, and similar devices. Electronic device 102, display 132, and AR device 134 may perform such functions as accepting and/or receiving user input, dynamically displaying one or more images, etc. The graphical user interface may be provided by various operating systems known in the art, such as, for example, iOS, Android, Windows Mobile, Windows, Mac OS, Chrome OS, Linux, Unix, USENIX, Phantom, a gaming platform OS (e.g., Xbox, PlayStation, Wii), and/or other operating systems.

In various embodiments, communication media 104 may be based on one or more wireless communication protocols such as Bluetooth®, ZigBee, 802.11 protocols, Infrared (IR), Radio Frequency (RF), 2G, 3G, 4G, 5G, and/or wired protocols and media. Communication media 104 may be implemented as a single medium in some cases.

As mentioned, electronic device 102 may take a variety of forms, such as a desktop or laptop computer, a smartphone, a tablet, a smartwatch or other wearable electronic device, a television or other audio or visual entertainment device or system, a camera (including still shot or video) or the like. Electronic device 102, display 132, and AR device 134 may communicate with other devices and/or with one another over communication media 104 with or without the use of server system 106. In various embodiments, electronic device 102, display 132, AR device 134, and/or server system 106 may be used to perform various processes described herein and/or may be used to execute various operations described herein with regard to one or more disclosed systems and methods. Upon studying the present disclosure, it will be appreciated that environment 100 may include multiple electronic devices 102, communication media 104, server systems 106, probe 126, display 132, AR devices 134, processing circuitry 138, and/or database 142.

As mentioned, communication media 104 may be used to connect or communicatively couple electronic device 102, display 132, AR device 134, and/or server system 106 to one another or to a network, and communication media 104 may be implemented in a variety of forms. For example, communication media 104 may include an Internet connection, such as a local area network (LAN), a wide area network (WAN), a fiber optic network, internet over power lines, a hard-wired connection (e.g., a bus), and the like, or any other kind of network connection. Communication media 104 may be implemented using any combination of routers, cables, modems, switches, fiber optics, wires, radio (e.g., microwave/RF links), and the like. Further, communication media 104 may be implemented using various wireless standards, such as Bluetooth, Wi-Fi, 3GPP standards (e.g., 2G GSM/GPRS/EDGE, 3G UMTS/CDMA2000, 4G LTE/LTE-U/LTE-A, 5G). Upon reading the present disclosure, it will be appreciated that there are other ways to implement communication media 104 for communications purposes.

Likewise, though not shown, it will be appreciated that a similar communication medium may be used to connect or communicatively couple position tracking 136, processing circuitry 138, and/or database 142 to one another, in addition to other elements of environment 100. In example embodiments, communication media 104 may be, or include, a wired or wireless wide area network (e.g., cellular, fiber, and/or circuit-switched connection) for electronic device 102, display 132, AR device 134, and/or server system 106, which may be relatively geographically disparate; and in some cases, aspects of communication media 104 may involve a wired or wireless local area network (e.g., Wi-Fi, Bluetooth, unlicensed wireless connection, USB, HDMI, and/or standard AV), which may be used to communicatively couple aspects of environment 100 that may be relatively close, geographically. In some embodiments, server system 106 may be remote from electronic device 102, display 132, and AR device 134,.

Server system 106 may provide, receive, collect, or monitor information from electronic device 102, display 132, and AR device 134, such as, for example, one or more images, user input, supplemental information, position information, orientation information depth information, metadata, and the like. Server system 106 may be configured to receive or send such information via communication media 104. This information may be stored in database 142 and may be processed using processing circuitry 138. For example, processing circuitry 138 may include an analytics engine capable of performing analytics on information that server system 106 has collected, received, or otherwise interacted with, from electronic device 102, display 132, or AR device 134. In embodiments, database 142, and processing circuitry 138 may be implemented as a distributed computing network or as a relational database or the like.

Server 108 may include, for example, an Internet server, a router, a desktop or laptop computer, a smartphone, a tablet, a processor, a component, or the like, and may be implemented in various forms, including, for example, an integrated circuit or collection thereof, a printed circuit board or collection thereof, or in a discrete housing/package/rack or multiple of the same.

In embodiments, server 108 directs communications for electronic device 102, display 132, or AR device 134 over communication media 104. For example, server 108 may process and exchange messages for electronic device 102, display 132, or AR device 134 that correspond to one or more images, user input, supplemental information, position information, orientation information depth information, metadata, and/or other information. Server 108 may update information stored on electronic device 102, display 132, or AR device 134, for example, by delivering one or more images, user input, supplemental information, position information, orientation information depth information, metadata, and/or other information thereto. Server 108 may send/receive information to/from electronic device 102, display 132, or AR device 134 in real time or sporadically. Further, server 108 may implement cloud computing capabilities for electronic device 102, display 132, or AR device 134.

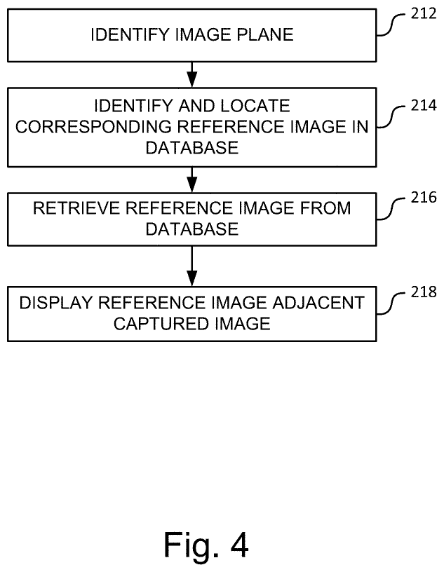

An example of this is illustrated in FIG. 4. Referring now to FIG. 4, at operation 212, the system identifies the image plane of the ultrasound system based on the current position and orientation of the ultrasound probe. At operation 214, the system identifies and locates the corresponding reference image in the database. For example, this is the reference image for the corresponding image plane from the “normal” subject. At operation 216, the reference image is retrieved from the database and provided to the processing system to be displayed to the operator. At operation 218, the reference image is displayed by the AR device along with the image captured of the subject by the ultrasound probe. In some embodiments, the images can be displayed side-by-side so that the operator can compare the subject image to the reference image. In other embodiments, the images can be overlapped or the AR device can toggle the display between the reference image in the subject image. In still further embodiments, notations, data and other information can be overlaid onto the display.

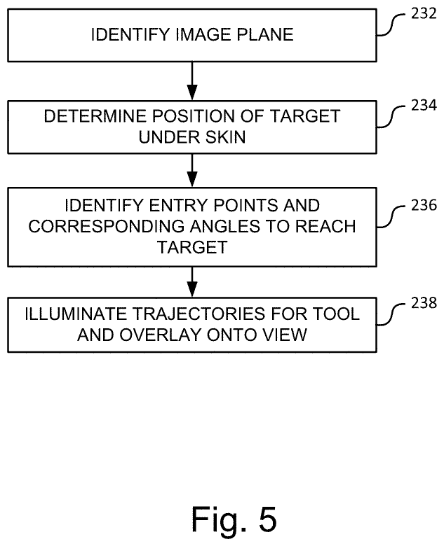

In yet further embodiments, the system can be used to aid in various procedures performed on the subject. For example, in some embodiments, the system can be configured to aid the practitioner by illustrating paths or trajectories for needles or other tools. An example of this is illustrated in FIG. 5. Referring now to FIG. 5, at operation 232 the system identifies the imaging plane of the ultrasound system based on the current position and orientation of the ultrasound probe. At operation 234, the system determines the position of a target tissue beneath the skin of the subject. For example, the target tissue can be a tissue at which a surgical implement is to be positioned. The target tissue can be identified, for example, by an operator in the system can use a positioning information of the probe to determine the location of the target in 3D space, including the depth of the target beneath the skin of the subject.

Based on the determined location of the target, the system can identify entry points and corresponding angles for insertion of the surgical implement to reach the identified target. The system may identify entry points using image recognition to determine spaces between objects, using machine-learning techniques to identify successful past entry points and trajectories, by presenting or displaying one or more images to an operator, or other methods. This is illustrated at operation 236. Depending on the target and the subject, there may be multiple entry points and corresponding angles based on the entry points. At operation 238, the system displays the entry points and trajectories for the tool on the AR device. In some embodiments, the entry points and trajectories can be overlaid onto the displayed image. In some embodiments, an operator may input one or more marks on the virtual displayed image to identify entry points and trajectories. The one or more marks may be overlaid onto the displayed image. During the insertion or other operation of the implement, the actual position of the implement can be compared with the overlays to help guide the operator.

In yet further embodiments, 3D reconstructions can be made using captured image slices and information identifying the position of each scan. For example, the system can be configured such that when an operator moves the ultrasound probe across a section of the subject, the image slices are captured along with position information for each image slice. The slices, the position of the slice, and the depth information in the captured image can be used to re-create a 3D image of the portion of the subject that was scanned.

In the various elements and components of the enhanced ultrasound imaging system can be connected via wired or wireless communication links. For example, in some embodiments, the ultrasound probe can be coupled to the other components via a Wi-Fi or other wireless communication link.

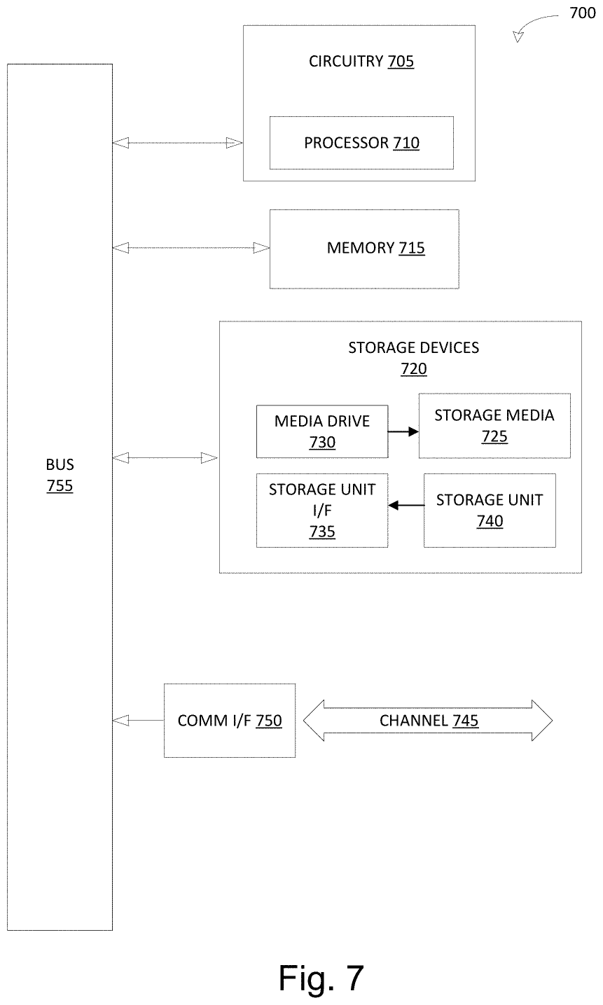

FIG. 7 illustrates example computing component 700, which may in some instances include a processor/controller resident on a computer system (e.g., server system 106, electronic device 102, display 132, or AR device 134). Computing component 700 may be used to implement various features and/or functionality of embodiments of the systems, devices, and methods disclosed herein. With regard to the above-described embodiments set forth herein in the context of systems, devices, and methods described with reference to FIGS. 1 through 6, including embodiments involving electronic device 102, probe 126, display 132, AR device, 134, position tracking 136, and/or processing circuitry 138, it may be appreciated additional variations and details regarding the functionality of these embodiments that may be carried out by computing component 700. In this connection, it will also be appreciated upon studying the present disclosure that features and aspects of the various embodiments (e.g., systems) described herein may be implemented with respect to other embodiments (e.g., methods) described herein without departing from the spirit of the disclosure.

As used herein, the term component may describe a given unit of functionality that may be performed in accordance with one or more embodiments of the present application. As used herein, a component may be implemented utilizing any form of hardware, software, or a combination thereof. For example, one or more processors, controllers, ASICs, PLAs, PALs, CPLDs, FPGAs, logical components, software routines, or other mechanisms may be implemented to make up a component. In embodiments, the various components and circuits described herein may be implemented as discrete components or the functions and features described may be shared in part or in total among two or more components. In other words, it should be appreciated that after reading this description, the various features and functionality described herein may be implemented in any given application and may be implemented in one or more separate or shared components in various combinations and permutations. Even though various features or elements of functionality may be individually described or claimed as separate components, it will be appreciated that upon studying the present disclosure that these features and functionality may be shared among one or more common software and hardware elements, and such description shall not require or imply that separate hardware or software components are used to implement such features or functionality.

Where components of the application are implemented in whole or in part using software, in embodiments, these software elements may be implemented to operate with a computing or processing component capable of carrying out the functionality described with respect thereto. One such example computing component is shown in FIG. 7. Various embodiments are described in terms of example computing component 700. After reading this description, it will be appreciated how to implement example configurations described herein using other computing components or architectures.

Referring now to FIG. 7, computing component 700 may represent, for example, computing or processing capabilities found within mainframes, supercomputers, workstations or servers; desktop, laptop, notebook, or tablet computers; hand-held computing devices (tablets, PDA's, smartphones, mobile phones, palmtops, etc.); or the like, depending on the application and/or environment for which computing component 700 is specifically purposed.

Computing component 700 may include, for example, one or more processors, controllers, control components, or other processing devices, such as a processor 710, and such as may be included in circuitry 705. Processor 710 may be implemented using a special-purpose processing engine such as, for example, a microprocessor, controller, or other control logic. In the illustrated example, processor 710 is connected to bus 755 by way of circuitry 705, although any communication medium may be used to facilitate interaction with other components of computing component 700 or to communicate externally.

Computing component 700 may also include one or more memory components, simply referred to herein as main memory 715. For example, main memory 715 may include random access memory (RAM) or other dynamic memory may be used for storing information and instructions to be executed by processor 710 or circuitry 705. Main memory 715 may also be used for storing temporary variables or other intermediate information during execution of instructions to be executed by processor 710 or circuitry 705. Computing component 700 may likewise include a read only memory (ROM) or other static storage device coupled to bus 755 for storing static information and instructions for processor 710 or circuitry 705.

Computing component 700 may also include one or more various forms of information storage devices 720, which may include, for example, media drive 730 and storage unit interface 735. Media drive 730 may include a drive or other mechanism to support fixed or removable storage media 725. For example, a hard disk drive, a floppy disk drive, a solid-state drive, a magnetic tape drive, an optical disk drive, a CD or DVD drive (R or RW), a Blu-ray drive, or other removable or fixed media drive may be provided. Accordingly, removable storage media 725 may include, for example, a hard disk, a floppy disk, a solid-state drive, magnetic tape, cartridge, optical disk, a CD, DVD, Blu-ray, or other fixed or removable medium that is read by, written to, or accessed by media drive 730. As these examples illustrate, removable storage media 725 may include a computer usable storage medium having stored therein computer software or data.

In alternative embodiments, information storage devices 720 may include other similar instrumentalities for allowing computer programs or other instructions or data to be loaded into computing component 700. Such instrumentalities may include, for example, fixed or removable storage unit 740 and storage unit interface 735. Examples of such removable storage units 740 and storage unit interfaces 735 may include a program cartridge and cartridge interface, a removable memory (for example, a flash memory or other removable memory component) and memory slot, a PCMCIA slot and card, and other fixed or removable storage units 740 and storage unit interfaces 735 that allow software and data to be transferred from removable storage unit 740 to computing component 700.

Computing component 700 may also include a communications interface 750. Communications interface 750 may be used to allow software and data to be transferred between computing component 700 and external devices. Examples of communications interface 750 include a modem or softmodem, a network interface (such as an Ethernet, network interface card, WiMedia, IEEE 802.XX, or other interface), a communications port (such as for example, a USB port, IR port, RF port, RS232 port Bluetooth® interface, or other port), or other communications interface. Software and data transferred via communications interface 750 may be carried on signals, which may be electronic, electromagnetic (which includes optical) or other signals capable of being exchanged by a given communications interface 750. These signals may be provided to/from communications interface 750 via channel 745. Channel 745 may carry signals and may be implemented using a wired or wireless communication medium. Some non-limiting examples of channel 745 include a phone line, a cellular or other radio link, an RF link, an optical link, a network interface, a local or wide area network, and other wired or wireless communications channels.

In this document, the terms “computer program medium,” “machine readable medium,” and “computer usable medium” are used to generally refer to transitory or non-transitory media such as, for example, main memory 715, storage unit interface 735, removable storage media 725, and channel 745. These and other various forms of computer program media, computer readable media, or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions may enable the computing component 700 or a processor to perform features or functions of the present application as discussed herein.

Various embodiments have been described with reference to specific example features thereof. It will, however, be evident that various modifications and changes may be made thereto without departing from the broader spirit and scope of the various embodiments as set forth in the appended claims. The specification and figures are, accordingly, to be regarded in an illustrative rather than a restrictive sense.

Although described above in terms of various example embodiments and implementations, it should be understood that the various features, aspects, and functionality described in one or more of the individual embodiments are not limited in their applicability to the particular embodiment with which they are described, but instead may be applied, alone or in various combinations, to one or more of the other embodiments of the present application, whether or not such embodiments are described and whether or not such features are presented as being a part of a described embodiment. Thus, the breadth and scope of the present application should not be limited by any of the above-described example embodiments.

Terms and phrases used in the present application, and variations thereof, unless otherwise expressly stated, should be construed as open ended as opposed to limiting. As examples of the foregoing: the term “including” should be read as meaning “including, without limitation,” or the like; the term “example” is used to provide illustrative instances of the item in discussion, not an exhaustive or limiting list thereof; the terms “a” or “an” should be read as meaning “at least one,” “one or more,” or the like; and adjectives such as “standard,” “known,” and terms of similar meaning should not be construed as limiting the item described to a given time period or to an item available as of a given time, but instead should be read to encompass standard technologies that may be available or known now or at any time in the future. Likewise, where this document refers to technologies that would be appreciated to one of ordinary skill in the art, such technologies encompass that which would be appreciated by the skilled artisan now or at any time in the future.

The presence of broadening words and phrases such as “one or more,” “at least,” “but not limited to,” or other like phrases in some instances shall not be read to mean that the narrower case is intended or required in instances where such broadening phrases may be absent. The use of the term “component” does not imply that the components or functionality described or claimed as part of the component are all configured in a common package. Indeed, any or all of the various components of a component, whether control logic or other components, may be combined in a single package or separately maintained and may further be distributed in multiple groupings or packages or across multiple locations.

Additionally, the various embodiments set forth herein are described in terms of example block diagrams, flow charts, and other illustrations. As will be appreciated after reading this document, the illustrated embodiments and their various alternatives may be implemented without confinement to the illustrated examples. For example, block diagrams and their accompanying description should not be construed as mandating a particular architecture or configuration.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. An ultrasound imaging system, comprising:
an ultrasound probe;
processing circuitry communicatively coupled to the ultrasound probe; and
an AR device receiving image information from the processing circuitry and displaying one or more ultrasound images from the ultrasound probe in a field of view of an operator.

2. The system of claim 1, wherein the ultrasound probe comprises a tracking mechanism to generate and send position information and orientation information of the ultrasound probe to the AR device, such that the one or more ultrasound images dynamically move as a position of the ultrasound probe changes.

3. The system of claim 1, wherein the AR device further receives depth information corresponding to the one or more ultrasound image.

4. The system of claim 3, wherein the ultrasound probe comprises a first input, such that interacting with the first input changes a depth of the one or more ultrasound images displayed in the field of view of the operator.

5. The system of claim 1, wherein the ultrasound probe comprises a second input, such that interacting with the second input freezes one of the one or more ultrasound images.

6. The system of claim 1, wherein the ultrasound probe further receives supplemental information and displays the supplemental information in the field of view of the operator.

7. The system of claim 6, wherein the supplemental information comprises one or more of patient information, treatment information, medication information, and a reference ultrasound image.

8. The system of claim 1, wherein a given ultrasound image comprises:
depth information for the given ultrasound image; and
position information for the given ultrasound image, such that the one or more ultrasound images and corresponding depth and position information are used to generate a 3D image of a region captured by the ultrasound probe.

9. A method for ultrasound imaging, comprising:
receiving position and orientation information from an ultrasound probe;
receiving image information on a current image captured by the ultrasound probe;
identifying an imaging plane for the current image captured by the ultrasound probe based on the position and orientation information;
retrieving a reference image from a database, the reference image corresponding to the imaging plane of the current image captured by the ultrasound probe; and
sending the current image and the reference image to an AR device to display the current image and the reference image in an operator's field of view.

10. The method of claim 9, wherein the current image is displayed in the operator's field of view such that the current image appears to project from a distal end of the ultrasound probe.

11. The method of claim 9, wherein the current image is displayed adjacent to the reference image.

12. The method of claim 9, wherein the ultrasound probe further receives supplemental information and displays the supplemental information in the operator's field of view.

13. The method of claim 13, wherein the supplemental information comprises one or more of patient information, treatment information, medication information, and a reference ultrasound image.

14. The method of claim 13, wherein the supplemental information is overlaid onto the current image.

15. A method for ultrasound imaging, comprising:
receiving position and orientation information from an ultrasound probe;
receiving image information on a current image captured by the ultrasound probe;
identifying an imaging plane for a current image captured by the ultrasound probe based on the position and orientation information;
sending the current image to an AR device to display the current image in a field of view of an operator; and
determining a position of a target under a portion of skin of a subject.

16. The method of claim 15, further comprising:
identifying an entry point and a corresponding trajectory from the entry point for a tool to reach the target; and
sending the entry point and the corresponding trajectory to the AR device to overlay an image of the entry point and the corresponding trajectory onto the current image in the field of view of the operator.

17. The method of claim 16, further comprising:
receiving position and orientation information from the tool; and
sending the position and orientation information corresponding to the tool to the AR device to overlay an image of the tool onto the entry point and the corresponding trajectory overlaid onto the current image in the field of view of the operator.

18. The method of claim 15, further comprising sending a position of the target to the AR device to display the position in the field of view of the operator.

19. The method of claim 15, further comprising extracting depth information from the current image captured by the ultrasound probe.

20. The method of claim 15, wherein the target is tissue.
